(19) United States
(12) Patent Application Publication
(10) Pub. No.: US 2010/0178316 A1
(43) Pub. Date: Jul. 15, 2010

(54) EXTENDED RELEASE OF BIOACTIVE MOLECULES FROM SILICONE HYDROGELS

(76) Inventors: Anuj Chauhan, Gainesville, FL (US); Jinah Kim, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/602,322

(22) PCT Filed: May 30, 2008
(86) PCT No.: PCT/US08/65325

§ 371 Date: Nov. 30, 2009

Related U.S. Application Data
(60) Provisional application No. 60/940,795, filed on May 30, 2007.

(51) Int. Cl. A61F 2/00; A61K 31/573; A61K 31/5377; A61P 27/02

(52) U.S. Cl. 424/429; 424/422; 514/180; 514/236.2

(57) ABSTRACT

A dosage form for the administration of a biologically active agent is a silicone hydrogel appliance where the agent is absorbed in a copolymer of at least one siloxane containing monomer and at least one hydrophilic monomer where the appliance is placed in contact with a tissue surface of the patient, such as a contact lens placed in the eye. The appliance can be formed where a copolymer of the siloxane containing and hydrophobic monomers is soaked in a solution of the biologically active agent. The agent can be a non-ionic drug which is absorbed into the appliance from a non-aqueous solution, such as an ethanol solution.

### CROSS-REFERENCE TO RELATED APPLICATION

The present application claims the benefit of U.S. Provisional Application Ser. No. 60/940,795, filed May 30, 2007, which is hereby incorporated by reference herein in its entirety, including any figures, tables, or drawings.

### FIELD OF THE INVENTION

The present invention relates to methods and systems for the ocular delivery of drugs and other bioactive molecules from silicone hydrogels to patients in need thereof.

### BACKGROUND

Providing and maintaining adequate concentrations of bioactive agents, such as drugs, for example, in the pre-corneal tear film for extended periods of time is one of the major problems plaguing methods and systems for ocular drug delivery. Topical delivery via eye drops, which accounts for about 90% of all ophthalmic formulations, is very inefficient and in some instances leads to serious side effects [Lang, “Ocular drug delivery conventional ocular formulations” Adv. Drug Delivery, 1995, 16: 39-43]. Only about 5% of the drug applied as drops penetrate through the cornea and reaches the ocular tissue, while the rest is lost due to tear drainage [Bourlais et al., “Ophthalmic drug delivery systems” Progress in Retinal and Eye Research, 1998, 17, 1: 33-58]. The drug mixes with the fluid present in the tear film upon instillation and has a short residence time of about 2-5 minutes in the film. About 5% of the drug gets absorbed and the remaining drug flows through the upper and the lower canaliculi into the lacrimal sac. The drug containing tear fluid is carried from the lacrimal sac into the nasolacrimal duct, and eventually, the drug gets absorbed into the bloodstream. This absorption leads to drug wastage and, more importantly, the presence of certain drugs in the bloodstream leads to undesirable side effects. Drainage of instilled drug with the tear fluid, and absorption through the conjunctiva leads to a short duration of action. Hence, an ophthalmic drug delivery system that will be as convenient as a drop but will increase the residence time of the drug in the eye and serve as a controlled release vehicle is highly desirable [Nagarsenker et al., “Preparation and evaluation of liposomal formulations of tropicamide for ocular delivery” Int. J. of Pharm., 1990, 190: 63-71].

A contact lens is an ideal vehicle for delivering drugs to the eye for a number of reasons. One reason is that present-day soft contact lenses can be worn comfortably and safely for an extended period of time, varying from about a day to 30 days. Once the contact lens is placed on the eye, the drug from the lens will diffuse into a thin fluid layer trapped between the lens and the cornea, namely the post-lens tear film (POLTF). There is limited mixing between the fluid in the POLTF and the outside tear fluid [Creech et al., “Dispersive mixing in the posterior tear film under a soft contact lens” Ind. Eng. Chem. Res., 2001, 40, 3015-26; Mc Namara et al., C. D. “Tear mixing under a soft contact lens: Effects of lens diameter” Am. J. of Ophth., 1999, 127, 659-65.]. Thus, the drug to be released from the lens will have a long residence time in the eye. Another advantage of this system is that it could simultaneously correct vision while it delivers one or more drugs to the cornea. The fraction of drug that enters the cornea when it is delivered via contact lenses is expected to be much higher than that possible from drops because the residence time of solutes in the tear film in between the contact lens and the cornea is about 30 minutes, which is significantly larger than the residence time of drugs delivered as drops. It has been shown by a general mathematical model that the bioavailability, which is the fraction of the applied drug that enters the cornea, could be as high as 50% for drugs delivered by contact lenses [Li et al., “Modeling ophthalmic drug delivery by soaked contact lenses” Ind. Eng. Chem. Res., 2006, 45, 3718-34.]

There have been a number of attempts in the past to use contact lenses for ophthalmic drug delivery; however most of these focused on soaking hydrophilic lenses in an aqueous drug solution followed by insertion into the eye [Hehl et al., “Improved penetration of aminoglycosides and fluoroquinolones into the aqueous humour of patients by means of Acuvue contact lenses” Eur. J. Clin. Pharmacol., 1999, 55, 317-23; Hillman, “Management of acute glaucoma with Pilocarpine-soaked hydrophilic lens” Brit. J. Ophthal., 1974, 58, 674-9; Ramer et al., “Ocular Penetration of Pilocarpine” Annals of Ophthalmology, 1974, 6, 1325-7; Montague et al., “Pilocarpine dispensation for the soft hydrophilic contact lens” Brit. J. Ophthal., 1975, 59, 455-8; Hillman et al., “Pilocarpine delivery by hydrophilic lens in the management of acute glaucoma” Transactions of the Ophthalmological Societies of the United Kingdom, 1975, 79-84; Giambattista et al., “Possibility of Isoproterenol Therapy with Soft Contact Lenses: Ocular Hypotension Without Systemic Effects” Ann. Ophthalmol., 1976, 8, 819-29; Marmion, “Pilocarpine administration by contact lens” Trans. Ophthal. Soc., U.K., 1977, 97, 162-3]. In one of the studies, Hehl et al., the lens are soaked in eye-drop solutions for one hour followed by lens insertion in the eye. Five different drugs were studied and it was concluded that the amount of drug released by the lenses are lower or of the same order of magnitude as the drug released by eye drops. This may occur because the maximum drug concentration obtained in the lens matrix is limited to the equilibrium concentration. Nakada et al., U.S. Pat. No. 6,027,745, May 29, 1998, discloses a contact lens with a hollow cavity by bonding together two separate pieces of lens material. The compound lens is soaked in the drug solution. The lens imbibes the drug solution and slowly releases it upon insertion in the eye. The compound lens suffers from the same limitations as the drug-soaked lens because the concentration of the drug in the cavity is the same as the concentration of the drug in the drug solution and thus such a lens can supply the drug for a limited amount of time. Furthermore, the presence of two separate sheets of lens material can lead to smaller oxygen and carbon dioxide permeabilities that can cause an edema in the corneal tissue. The other studies listed above, Hillman, Ramer et al., Montague et al., Hillman et al., Giambattista et al., and Marmion et al., and the patents, Rosenwald, U.S. Pat. No. 4,484,922, and “Schultz et al., U.S. Pat. No. 5,723,131, suffer from the same limitations because they are also based on soaking of hydrophilic contact lenses or similar devices in aqueous drug-solutions followed by insertion into the eye. An additional problem that can occur when absorbing drugs in hydrophilic contact lens, Schultz et al., U.S. Pat. No. 6,410,045, is that the preservatives included in the drug are often preferentially absorbed in the lens and the preservative can be selectively absorbed to a level that is toxic while that of the drug may be below effective levels.

A number of references: Elisseeff et al., “Photoencapsulation of chondrocytes in poly(ethylene oxide)-based semi-interpenetrating networks” Journal of Biomedical Materials Research, 2000, 51, 2, 164-71; Ward et al., “Preparation of controlled release systems by free-radical UV polymerizations in the presence of a drug” Journal of Controlled Release, 2001, 71, 2, 183-92; Scott et al., “Highly cross-linked, PEG-containing copolymers for sustained solute delivery” Biomaterials, 1999, 20, 15, 1371-80; Podual et al., “Preparation and dynamic response of cationic copolymer hydrogels containing glucose oxidase” Polymer, 2000, 41, 11, 3975-83; Colombo et al., “Observation of swelling process and diffusion front position during swelling in hydroxypropyl methyl cellulose (HPMC) matrices containing a soluble drug” Journal of Controlled Release, 1999, 61, 1, 2, 83-91; and Ende et al., “Transport of ionizable drugs and proteins in cross-linked poly(acrylic acid) and poly(acrylic acid-co-2-hydroxyethyl methacrylate) hydrogels. 2. Diffusion and release studies” Journal of Controlled Release, 1997, 48, 1, 47-56, have polymerized monomers that comprise a hydrogel in presence of the encapsulated proteins, cells and drugs to entrap these species in a hydrogel matrix. Although such direct loading of drug into the lenses can permit higher loadings of the drugs, it can result in an activity loss during polymerization.

A majority of the drug can diffuse from the lenses into the packaging medium and the drug retained in the lens can diffuse from the lens rapidly after insertion into the eye. To address these issues, Gulsen et al., “Dispersion of microemulsion drops in HEMA hydrogel: a potential ophthalmic drug delivery vehicle” Int. J. Pharm., 2005, 292, 95-117, and Gulsen et al., “Ophthalmic drug delivery through contact lenses” Invest. Ophth. Vis. Sci., 2004, 45, 2342-47, has proposed the development of nanoparticle laden gels that can load substantial amount of drug to the gel which can be released at a controlled rate from the nanoparticles. Also Graziacascone et al., “Poly(vinyl alcohol) hydrogels as hydrophilic matrices for the release of lipophilic drugs loaded in PLGA nanoparticles” Journal of Material Science: Materials in Medicine, 2002, 13, 29-32, discloses a study on encapsulating lipophilic drugs inside nanoparticles, and entrapping the particles in hydrogels. PVA hydrogels were used as hydrophilic matrices for the release of lipophilic drugs loaded in PLGA particles. These systems display the shortcoming of burst release due to the presence of the drag outside the particles. A number of references: Hiratani et al., “The nature of backbone monomers determines the performance of imprinted soft contact lenses as timolol drug delivery systems” Biomaterials, 2004, 25, 1105-13; Hiratani et al., “Ocular release of timolol from molecularly imprinted soft contact lenses” Biomaterials, 2005, 26, 1293-8; Hiratani et al., “Controlling drug release from imprinted hydrogels by modifying the characteristics of the imprinted cavities” Macromol. Biosci., 2005, 5, 728-33; Alverez-Lorenzo et al., “Soft contact lenses capable of sustained delivery of timolol” J. Pharm. Sci., 2002, 91, 2182-92; Hiratani et al., “Timolol uptake and release by imprinted soft contact lenses made of N,N-diethylacrylamide and methacrylic acid” J. Control Release, 2002, 83, 223-30, have focused on developing ‘imprinted’ contact lenses. The imprinting leads to an increase in the partition coefficients and slower release of drugs, but the increase is not very substantial, and these lenses typically have an initial burst release.

The studies listed above focused on the use of hydrophilic contact lenses for drug release. Recently Karlgard et al., “In vitro uptake and release studies of ocular pharmaceutical agents by silicon-containing and p-HEMA hydrogel contact lens materials” Int. J. Pharmaceutics, 2003, 257, 141-51 measured the uptake and release of a number of ophthalmic drugs by commercially available HEMA based and silicone contact lenses in vitro studies. A drug, either cromolyn sodium, ketotifen fumarate, ketorolac tromethamine or dexamethasone sodium phosphate, was loaded into these lenses by soaking these in aqueous drug solutions for a limited period of time. The release studies showed that a majority of the drug taken up by the gels was released in a short period of time. The silicone containing lenses were inferior to p-HEMA containing hydrogel lenses but could release equivalent amounts to those delivered by a single 50 μL eyedrop solution assuming a 7 μL tear volume.

In all the studies listed above, the lenses deliver ophthalmic drugs for a limited period of time ranging from a few hours to a few days. However, the practical use of such drug delivery systems require extended-wear lenses capable of releasing ophthalmic drugs without a burst release and for a period of about a week to about a month or more.

### SUMMARY OF THE INVENTION

The invention is directed to a method of controlled release of a bioactive agent from a silicone hydrogel appliance, such as a contact lens, where a silicone hydrogel article has at least one bioactive agent absorbed into the article, to form the appliance, which is placed onto a tissue surface, such as an eye, to treat a patient. The release occurs over a period in excess of one week and with no initial burst release of drug occurs. The drug is released at an approximately constant rate over the majority of the time of release. The bioactive agent can be a drug. In one embodiment of the invention the drug is a non-ionic drug such as dexamethasone (DX), dexamethasone acetate (DXA) and timolol. In one embodiment of the invention, the silicone hydrogel article is a copolymer of at least one siloxane containing monomer and at least one hydrophilic monomer. Exemplary siloxane containing monomers are 3-methacryloxy-propyltris(trimethylsiloxy)silane (TRIS) and a siloxane macromer that has acrylate or methacrylate units. Exemplary hydrophilic monomers are N,N-dimethylacrylamide (DMA) and 1-vinyl-2-pyrrolidone (NVP). In an embodiment of the invention, the hydrophilic monomers can be 10 to 50 percent of the copolymer.

In an embodiment of the invention, at least one monomer in the copolymer can provide functionality for specific interaction with at least one bioactive agent. The functionality can be an ionic functionality, a metal complexing functionality, a multiple hydrogen bonding functionality, or a functionality that mimics a biological receptor for a bioactive agent.

The bioactive agent can be absorbed into the silicone hydrogel article by soaking the article in a solution containing the agent for a period of time that has been determined to be appropriate to achieve a desired level of the agent in the appliance. The solution includes the bioactive agent and a solvent that can swell the silicone hydrogel article. A useful solvent for the solution is ethanol. In one embodiment of the invention, the solution is an aqueous solution and the silicone hydrogel article is soaked for about 1 to about 8 weeks.

The bioactive agent can be absorbed into mixture of the siloxane containing and hydrophilic monomers with a polymerization initiator in a mold and the initiation can be used to generate the hydrogel appliance.

The invention is also directed to a sustained-release dosage form useful for administration of a bioactive agent to a tissue surface where a silicone hydrogel article contains the bioactive agent in a therapeutically effective amount. The dosage form can be a silicone hydrogel contact lens for ocular administration of the bioactive agent, which can be a drug. In one embodiment of the dosage form, the silicone hydrogel article is 20 to 80 weight percent silicone. In an embodiment the drug can be a non-ionic drug, such as dexamethasone (DX), dexamethasone acetate (DXA) and timolol.

### BRIEF DESCRIPTION OF THE FIGURES

The embodiments of the invention illustrated in the figures do not limit the invention to that illustrated.

FIG. 1 shows the release of DXA according to an embodiment of the invention from a 0.4 mm thick by 1.5 cm by 1.5 cm sample of GEL1 into a phosphate buffered saline (PBS) solution (6 mL) over time where the PBS solution is exchanged at 360 hours into the extraction.

FIG. 2 shows the release of DX according to an embodiment of the invention from a 0.1 and 0.2 mm thick by 1.5 cm by 1.5 cm sample of GEL1 into a PBS solution (1.5 and 3 mL respectively) over time.

FIG. 3 shows the release of DX according to an embodiment of the invention from a 0.1 and 0.2 mm thick by 1.5 cm by 1.5 cm sample of GEL4 into a PBS solution (1.5 and 3 mL respectively) over time.

FIG. 4 shows the release of timolol according to an embodiment of the invention, loaded from a 0.64 mg/mL ethanol solution and subsequently dried, from a 0.1, 0.2, and 0.4 mm thick by 1.5 cm by 1.5 cm sample of GEL1 into a PBS solution (1.5, 3, and 6 mL respectively) over time, where the PBS solution is exchanged at 168 hours into the extraction.

FIG. 5 shows the release of timolol according to an embodiment of the invention, loaded from a 0.64 mg/mL ethanol solution and subsequently dried, from a 0.1 and 0.2 mm thick by 1.5 cm by 1.5 cm sample of GEL2 into a PBS solution (1.5 and 3 mL respectively) over time, where the PBS solution is exchanged at 1009 hours into the extraction for the 0.1 mm thick sample.

FIG. 6 shows the release of timolol according to an embodiment of the invention, loaded from a 0.64 mg/mL ethanol solution and subsequently dried, from 0.1, 0.2, and 0.4 mm thick by 1.5 cm by 1.5 cm samples of GEL3 into a PBS solution (1.5, 3, and 6 mL respectively) over time, where the PBS solution is exchanged at 1009 hours into the extraction for the 0.1 mm thick sample.

FIG. 7 shows the release of timolol according to an embodiment of the invention, loaded from a 0.64 mg/mL ethanol solution and subsequently dried, from 0.1, 0.2 and 0.4 mm thick by 1.5 cm by 1.5 cm samples of GEL4 into a PBS solution (1.5, 3, and 6 mL respectively) over time where the PBS solution is exchanged at 168 hours and 1009 hours into the extraction for the 0.2 mm and the 0.1 mm thick samples, respectively.

FIG. 8 shows the release of timolol according to an embodiment of the invention, loaded from a 6.4 mg/mL ethanol solution and subsequently dried, from a 0.4 mm thick by 1.5 cm by 1.5 cm sample of GEL1 into a PBS solution (6 mL) over time where the PBS solution is exchanged at 336 hours into the extraction.

FIG. 9 shows the release of timolol according to an embodiment of the invention, loaded from a 6.4 mg/mL ethanol solution and subsequently dried, from a 0.4 mm thick by 1.5 cm by 1.5 cm sample of GEL2 into a PBS solution (6 mL) over time where the PBS solution is exchanged at 552 hours into the extraction.

FIG. 10 shows the release of timolol according to an embodiment of the invention, loaded from a 6.4 mg/mL ethanol solution and subsequently dried, from a 0.4 mm thick by 1.5 cm by 1.5 cm sample of GEL3 into a PBS solution (6 mL respectively) over time.

FIG. 11 shows the uptake of DX according to an embodiment of the invention by a 0.1 mm thick by 1.5 cm by 1.5 cm sample of GEL1 from a 1.5 mL of PBS solution (0.026, 0.052, and 0.078 mg/mL respectively) over time.

FIG. 12 shows the uptake of DX according to an embodiment of the invention by a 0.1 mm thick by 1.5 cm by 1.5 cm sample of GEL4 from a 1.5 mL of PBS solution (0.026, 0.052, and 0.078 mg/mL respectively) over time.

FIG. 13 shows the uptake of timolol according to an embodiment of the invention by a 0.1 mm thick by 1.5 cm by 1.5 cm sample of GEL1 from a 1.5 mL of PBS solution (0.035, 0.070, and 0.105 mg/mL respectively) over time.

FIG. 14 shows the uptake of timolol according to an embodiment of the invention by a 0.1 mm thick by 1.5 cm by 1.5 cm sample of GEL2 from a 1.5 mL of PBS solution (0.035, 0.070, and 0.105 mg/mL respectively) over time.

FIG. 15 shows the uptake of timolol according to an embodiment of the invention by a 0.1 mm thick by 1.5 cm by 1.5 cm sample of GEL3 from a 1.5 mL of PBS solution (0.070, and 0.105 mg/mL respectively) over time.

FIG. 16 shows the uptake of timolol according to an embodiment of the invention by a 0.1 mm thick by 1.5 cm by 1.5 cm sample of GEL4 from a 1.5 mL of PBS solution (0.035, 0.070, and 0.105 mg/mL respectively) over time.

FIG. 17 shows the release of timolol according to an embodiment of the invention to a PBS solution after storage 0.1 mm thick GEL1 packaged for 1.3 and 2 month in another PBS solution.

FIG. 18 shows the release of DXA according to an embodiment of the invention to a PBS solution after storage 0.1 mm thick GEL1 packaged for 1.5 and 2 month in another PBS solution.

### DESCRIPTION OF THE INVENTION

The present invention is directed to a system for the delivery of bioactive agents to a patient via a silicon hydrogel appliance. One embodiment of the invention is for the ocular delivery of drugs from a silicone hydrogel lens. Silicon hydrogel lenses have been developed and are commercially available. They incorporate a hydrophobic silicone portion such that the transmission of oxygen through the lens is adequate to retain the health of the cornea when worn for extended periods of time. The invention involves the absorption of one or more bioactive agents into a silicone hydrogel and the extended release of the absorbed agent to a patient by the contact of the silicone hydrogel to a tissue surface of the patient. Bioactive agents that can be used in the practice of the invention include drugs, vitamins, wound healing agents, and anti fouling agents that display sufficient partitioning between the silicone hydrogel appliance and the environment of application such that the bioactive agent can be released effectively from the appliance and delivered to the desired tissue. Although many bioactive agents exist that can be employed in the practice of the invention, other are not appropriate for the practice of the invention as they do not partition between the hydrogel and the tissue environment to be effective in treating the tissue. It is to be understood that where the term drug appears in this application in a general manner, any appropriate bioactive agent can be used, though not specifically a drug, for the practice of the invention.

It was discovered that the release of drugs from the silicone hydrogel occurs over a period of time that is conducive to effective drug therapy. The profile of the drug release depends on the composition of the silicone hydrogel and the properties of drug, but in general it occurs over a period in excess of 40 days and the release occurs at a relatively constant rate. Although the initial release is more rapid than the release at longer time periods, no initial burst release of a large proportion of the absorbed drug followed by little release occurs in the inventive delivery system. The drug therapy may be one where administration of the drug can be that of a few hours, for example a typical work day or night's sleep of eight hours, to 24 hours for a condition that can be effectively treated with a single dosage. Other treatments can require one day to a week, or one week to a month or more for a more chronic condition. The release from a given silicone hydrogel appliance need not be longer than the appropriate duration that the silicone hydrogel article, such as a contact lens, can be in exposed to the tissue, such as an eye, to be treated.

The silicone hydrogels articles can be prepared in a manner similar to that common to preparation of such networks, where hydrophobic silicon containing monomers are included into the formulation and the silicone monomer is copolymerized with monomers to provide hydrophilic character to the resulting network. Usually a silicone monomer that can undergo addition into the growing polymer at two sites is included. Such silicone hydrogels are non-homogeneous structures, often displaying discernable phase separation between a silicone rich phase and a hydrophilic monomer derived phase. Depending upon the nature of these gels, surface treatment is sometimes necessary to assure the surface is sufficiently hydrophilic even though these gels are designed to incorporate 20 to more than 80 percent by weight water. Surface treatment can include coating with a hydrophilic coating or plasma etching to convert the silicon surface into a hydroxy group rich silicate type surface.

Suitable silicone hydrogel materials include, without limitation, silicone hydrogels made from silicone macromers such as the polydimethylsiloxane methacrylated with pendant hydrophilic groups described in U.S. Pat. Nos. 4,259,467; 4,260,725 and 4,261,875; or the polydimethylsiloxane macromers with polymerizable functional described in U.S. Pat. Nos. 4,136,250; 4,153,641; 4,189,546; 4,182,822; 4,343,927; 4,254,248; 4,355,147; 4,276,402; 4,327,203; 4,341,889; 4,486,577; 4,605,712; 4,543,398; 4,661,575; 4,703,097; 4,740,533; 4,837,289; 4,954,586; 4,954,587; 5,034,461; 5,070,215; 5,260,000; 5,310,779; 5,346,946; 5,352,714; 5,358,995; 5,387,632; 5,451,617; 5,486,579; 5,962,548; 5,981,615; 5,981,675; and 6,039,913. The silicone hydrogels can also be made using polysiloxane macromers incorporating hydrophilic monomers such as those described in U.S. Pat. Nos. 5,010,141; 5,057,578; 5,314,960; 5,371,147 and 5,336,797; or macromers comprising polydimethylsiloxane blocks and polyether blocks such as those described in U.S. Pat. Nos. 4,871,785 and 5,034,461.

Among the silicone containing monomers which may be in the formulation of a silicone hydrogel of the present invention include oligosiloxanylsilylalkyl acrylates and methacrylates containing from 2-10 Si-atoms. Typical representatives include: tris(trimethylsiloxysilyl)propyl(meth)acrylate, triphenyldimethyl-disiloxanylmethyl(meth)acrylate, pentamethyl-disiloxanylmethyl(meth)acrylate, tert-butyl-tetramethyl-disiloxanylethyl(meth)acrylate, methyldi(trimethylsiloxy)silylpropyl-glyceryl(meth)acrylate; pentamethyldisiloxanylmethyl methacrylate; heptamethylcyclotetrasiloxy methyl methacrylate; heptamethylcyclotetrasiloxy-propyl methacrylate; (trimethylsilyl)-decamethylpentasiloxypropyl methacrylate; and dodecamethylpentasiloxypropyl methacrylate.

Other representative silicon-containing monomers which may be used for silicone hydrogels of the present invention includes silicone-containing vinyl carbonate or vinyl carbamate monomers such as: 1,3-bis[4-vinyloxycarbonyloxy)but-1-yl]tetramethyldisiloxane; 3-(trimethylsilyl)propyl vinyl carbonate; 3-(vinyloxycarbonylthio)propyl-[tris(trimethylsiloxy)silane]; 3-[tris(trimethylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxethyl vinyl carbonate; trimethylsilylethyl vinyl carbonate; and trimethylsilylmethyl vinyl carbonate. Polyurethane-polysiloxane macromonomers (also sometimes referred to as prepolymers), which have hard-soft-hard blocks like traditional urethane elastomers, may be used. Examples of such silicone urethanes that may be included in the formulations of the present invention are disclosed in a variety or publications, including Lai, “The Role of Bulky Polysiloxanylalkyl Methacrylates in Polyurethane-Polysiloxane Hydrogels,” Journal of Applied Polymer Science, Vol. 60, 1193-1199 (1996).

Suitable hydrophilic monomers, which may be used separately or in combination for the silicone hydrogels of the present invention non-exclusively include, for example: unsaturated carboxylic acids, such as methacrylic and acrylic acids; acrylic substituted alcohols, such as 2-hydroxyethylmethacrylate, 2-hydroxyethylacrylate (HEMA), and tetraethyleneglycol dimethacrylate (TEGDMA); vinyl lactams, such as N-vinyl pyrrolidone; vinyl oxazolones, such as 2-vinyl-4,4′-dimethyl-2-oxazolin-5-one; and acrylamides, such as methacrylamide and N,N-dimethylacrylamide (DMA). Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Hydrophilic monomers may be incorporated into such copolymers, including, methacrylic acid and 2-hydroxyethyl methacrylamide.

The proportions of the monomers can vary over a large extent. The polymerization mixtures can also include effective amounts of additives, initiators, photoinitiators, and/or catalysts and that the reaction can be conducted in the presence of a diluent. Activation of the initiation of polymerization can be by thermal or photochemical means. The polymerization can occur via any ionic, radical or group transfer mechanism.

It was discovered that it was difficult to load sufficient quantities of drugs by soaking the silicone hydrogels in aqueous solutions of the drugs, where soaking occurs for periods of less than about 24 hours. With hydrophobic drugs, the drugs limited solubility in water permits only a small amount of drug to be dissolved in the water which limits the amount that can be absorbed by the silicone hydrogel from the water solution. In contrast highly hydrophilic drugs that have a larger solubility in water, generally display a low solubility in a silicone hydrogel. It has been discovered that in a preferred embodiment of the invention, loading either hydrophobic or hydrophilic drugs into the silicone hydrogel readily occurs by soaking the silicone hydrogel in a non-aqueous solution of drug where the organic solvent used swells the silicone gel. Non-limiting examples of such organic solvents include: ethanol; ethyl acetate; butyl acetate isopropanol; n-propanol; DMSO; methanol; toluene; methylene chloride; and tetrahydrofuran. In general, the solvent should be one that has a low toxicity, is non-carcinogenic, and is non-mutanogenic or can be removed essentially in total from the silicone gel by means commonly employed by those skilled in the art. Many hydrophobic and hydrophilic drugs are soluble in ethanol and so this solvent is conveniently used to load both types of drugs into the gel. The solvents are generally, but not necessarily, removed prior to placement of the hydrogel into the ocular environment or other tissue to be treated. The solvent can be removed, in addition to other methods, as a volatile off-gassing from the hydrogel and can be assisted separately or by any combination of vacuum, heating, a gas stream.

In another embodiment of the invention, drugs can be loaded into the gel by soaking in aqueous solutions. In these cases it is generally necessary to perform the loading over an extended period of time varying from weeks to months rather than the periods of less than about 24 hours that have been employed in the art. The slow loading appears to result because silicone hydrogels do not swell appreciably in water, leading to small diffusivities. The small diffusivity, which permits extended release, leads to slow loading where the loading rates are generally comparable to the release rates. Loading in this fashion can be carried out where the silicon gel article is sealed in a container that is used for distribution to a patient. The absorption of the bioactive agent into the article can occur over a long period of time, which includes the time of distribution of the appliance to patients. Typically, a use date indicated on such a package with this container would include an initial use date as well as an expiration date such that a sufficient level, often a near equilibrium or equilibrium level, of the bioactive agent in the appliance is achieved. A silicone hydrogel appliance, regardless of the solvent used for loading the bioactive agent into the appliance, can be distributed in an aqueous solution of the bioactive agent to avoid extraction of the agent from the appliance by a solution employed in the container for its distribution.

Typical silicone hydrogel contact lenses are packaged in a PBS buffer. A packaging buffer solution can extract a portion of the bioactive agent from the appliance, although the quantity partitioned from the appliance is only a fraction of the initially loaded amount and the appliance remains an effective delivery vehicle for the bioactive agent. The buffer solution can effectively extract some bioactive agent such that the delivery of the agent to tissue displays a more uniform rate over the time of the appliance's use than would have occurred absent storage in a buffer solution. The quantity of the bioactive agent extracted into the packaging solution can be reduced by including the drug in the packaging solution. Hence in an embodiment of the invention, the silicone hydrogel appliance can be packaged in an aqueous solution, for example in a PBS buffer solution, of the bioactive agent so that the bioactive agent is maintained at an equilibrium level in the appliance which has been loaded with the agent prior to packaging. The appliance is then in a state where the amount of bioactive agent therein is maintained at a desired level until placed on the eye or other tissue to be treated with the agent.

An alternative embodiment according to the invention for the loading of the drugs into the silicone hydrogels is the inclusion of the drugs during the polymerization of monomers and macromers to prepare the silicone hydrogel appliance. For drugs that display little or no soluble in the monomer mixture, the addition of a solvent, such as those used for swelling of silicone hydrogels can be included during a solution polymerization where all monomers and the drug are miscible.

A drug in a non-ionic form can be used in an embodiment of the invention, but a non-ionic form is not required for all embodiments of the invention. Many drugs traditionally supplied in an ionic form can be acquired in or converted to the non-ionic equivalent prior to loading in the silicone hydrogel. Such non-ionic drugs include dexamethasone (DX), dexamethasone acetate (DXA) and timolol, which are used for the examples below; however, any drug that may be absorbed in a silicon gel and is appropriate for introduction to the eye or other tissue to be treated may be used. If desired, a drug can be converted into a more hydrophobic form by protecting a polar functionality such as an acid group, an alcohol, or an amine in a manner where they remain protected until released from the silicone hydrogel into the aqueous environment of the eye or in the presence of other moist tissue. In general, the drug displays partitioning into the silicone hydrogel from an aqueous environment but that the partitioning is not absolute, such that the drug may be released slowly to the aqueous environment, for example into the tear film adjacent to the hydrogel when the appliance is placed in the eye. Multiple drugs may be absorbed into a single silicon hydrogel appliance.

When hydrophilic bioactive agents are included in a silicone hydrogel, the affinity of the hydrogel for the agent can be enhanced by the addition of functionality to the silicone hydrogel that specifically interact with the bioactive agent. For the incorporation of ionic drugs, the functionality can be ionic such that the ion pairing of the drug with that functionality occurs. For example, a negatively charged functionality in the silicone hydrogel can pair with a positively charged drug. Other functionality that can be incorporated into the silicone hydrogel are those that can complex a metal ion containing bioactive agent, that can promote two or more specific hydrogen bonding associations with a specific bioactive agent, or that can mimic the biological binding site of the patient for the bioactive agent, for example the binding site of an enzyme. Other interactions for the enhanced binding to a specific bioactive agent can be used depending upon the nature of the bioactive agent as can be recognized by one skilled in the art. Appliances of this type can be referred to as templated or imprinted appliances.

### Methods and Materials

### Example 1

Silicone gels were synthesized by free radical bulk polymerization of the monomer using a photoinitiator. Individually, 3 mL of a various mixtures of 3-methacryloxypropyltris(trimethylsiloxy)silane (TRIS), bis-alph,omega-(methacryloxypropyl)polydimethylsiloxane (Macromer), and N,N-dimethylacrylamide (DMA) with were combined with 0.18 mL of 1-vinyl-2-pyrrolidone (NVP) and 15 μL of ethylene glycol dimethacrylate (EGDMA). The compositions of monomer mixtures are indicated in Table 1. The mixture was purged with bubbling nitrogen for 20 min to remove oxygen. To each mixture was added 8 μg of the photoinitiator 2,4,6-Trimethylbenzoyl-diphenyl-phosphineoxide (Darocur® TPO) with stirring for 5 min and immediately injected into a mold which is composed of two 5 mm thick glass plates separated by a plastic spacer of a desired thickness. The glass plates are coated with a thin film of FEP (a polymer of tetrafluoroethylene and hexafloropropylene). The spacer thickness was chosen to be 0.1, 0.2, or 0.4 mm. The mold was then placed on Ultraviolet transilluminiator UVB-10 (Ultra·Lum, Inc.) and the gel was cured by irradiating by UVB light (305 nm) for 40 min. The molded gels were cut in pieces with square shaped tops (about 1.5×1.5 cm) and dried in air overnight before further use.

TABLE 1
Compositions of monomer mixture

| | TRIS | Macromer | DMA | NVP | EGDMA |
|---|---|---|---|---|---|
| | [mL] | [mL] | [mL] | [mL] | [μL] |
| GEL1 | 2.0 | 0.5 | 0.5 | 0.18 | 15 |
| GEL2 | 2.14 | 0.43 | 0.43 | 0.18 | 15 |
| GEL3 | 2.4 | 0.3 | 0.3 | 0.18 | 15 |
| GEL4 | 1.71 | 0.86 | 0.43 | 0.18 | 15 |

Drugs were individually loaded into individual square pieces of the gels by soaking a gel in 2 or 2.5 mL of a drug-ethanol solution for a period of 3 hours. At the end of three hours the gel was taken out and excess drug-ethanol solution was blotted from the surface of the piece with wipes. The gels were dried in air overnight, and subsequently used for drug release experiments. Results of drug release experiment are given below for three drugs (dexamethasone (DX), dexamethasone acetate (DXA) and timolol) which were individually loaded into different gel pieces.

Drug release experiments were conducted by soaking the square shaped drug containing gel pieces, prepared as described in Example 1, in Dulbecco's phosphate buffered saline (PBS). The volume of the PBS was varied depending on the thickness of the gel to maintain a constant volume ratio of gel to PBS for all samples. The gels of 0.1 mm, 0.2 mm, and 0.4 mm thickness were soaked in 1.5 mL, 3 mL, 6 mL of PBS, respectively.

### Example 2

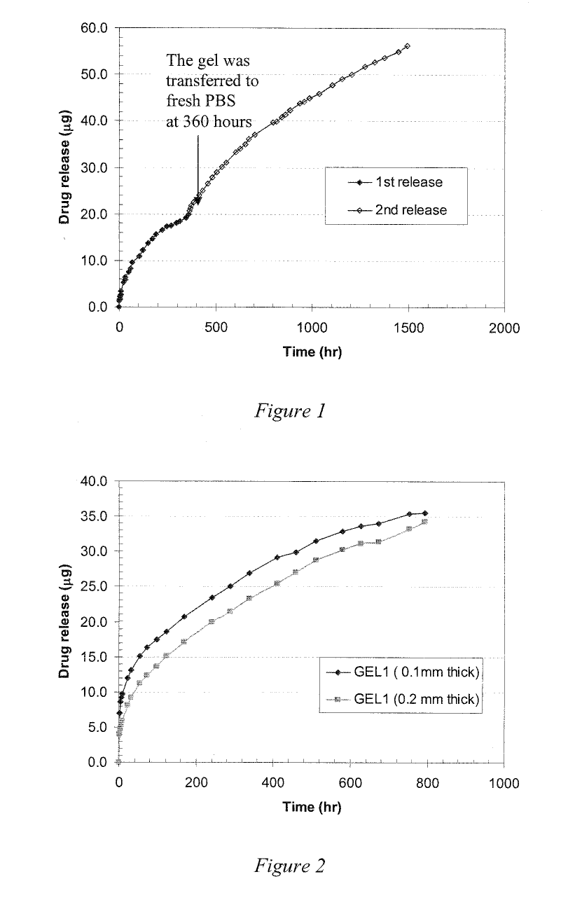

A DXA loaded 0.4 mm thick GEL1 piece was placed in 6 mL of PBS and the concentration change of DXA in the PBS solution was monitored. At 360 hours the gel was transferred to a fresh 6 mL PBS solution and monitoring of the change in concentration was continued. The total quantity of DXA level extracted from the gel piece is shown in FIG. 1.

### Example 2

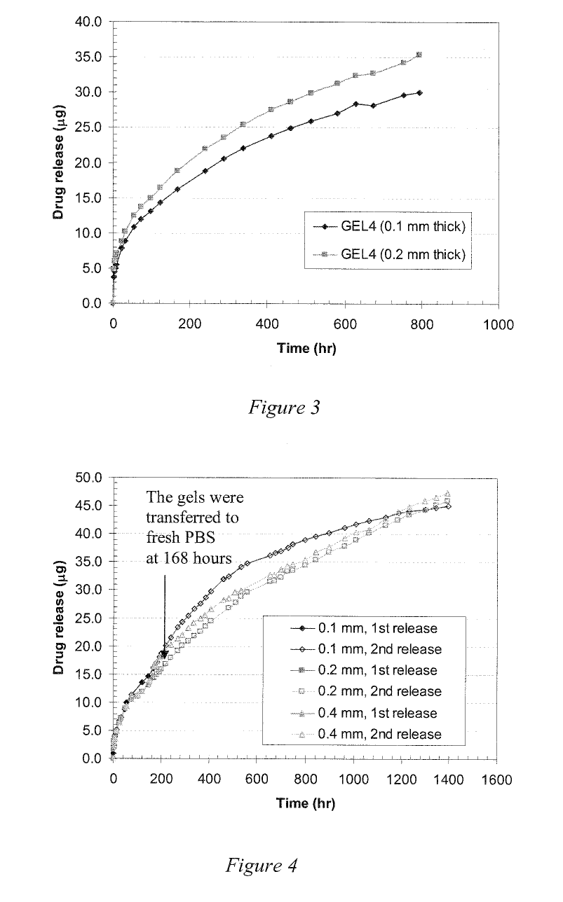

Two thicknesses, 0.1 and 0.2 mm, of DXA loaded GEL1 were prepared by soaking each separately in 2.5 mL of DX-ethanol solution (4.99 mg/mL) for 3 hours and were subsequently dried overnight. The DX loaded samples were separately placed in 6 mL of PBS and the concentration change of DX in the PBS solution was monitored, as shown in FIG. 2. In like manner, 0.1 and 0.2 thick samples of DX loaded GEL4 were placed in 6 mL of PBS and the concentration change observed is given in FIG. 3.

### Example 3

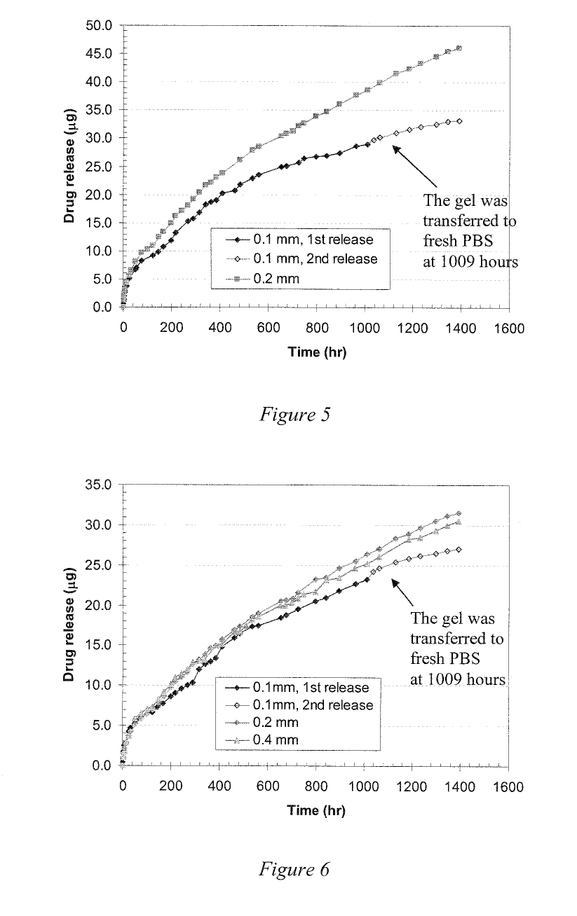

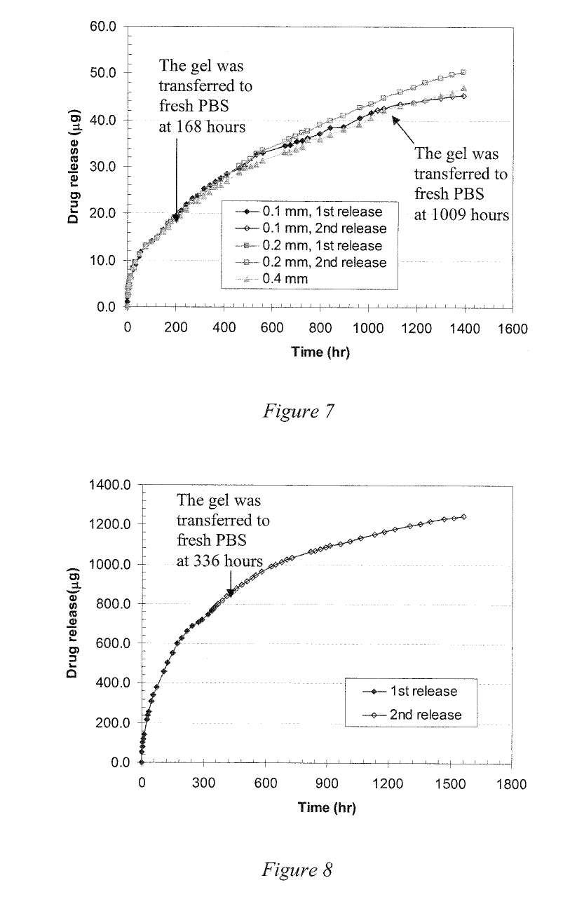

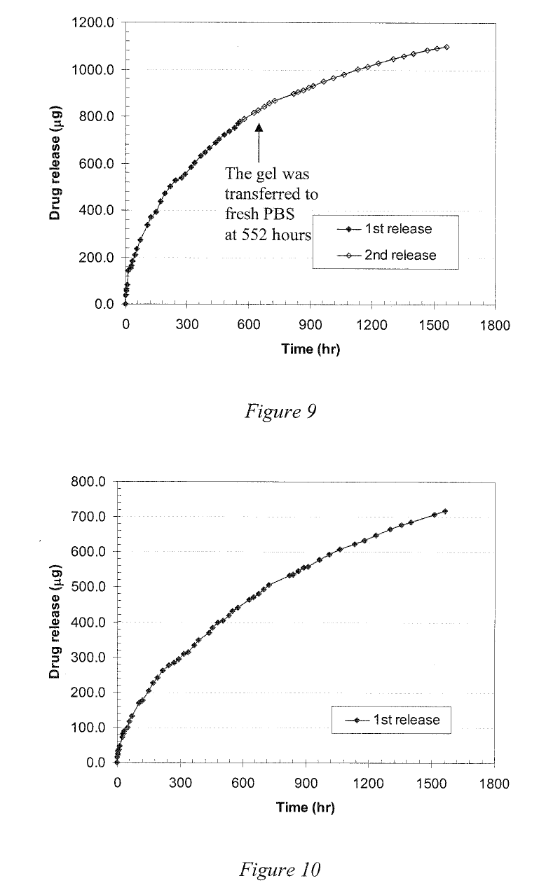

In like manner to Examples 1, 2 and 3, timolol release experiments were conducted with square pieces of the gels of three different thicknesses for the four different compositions prepared in Example 1 (GEL1 to GEL4), with two different drug loadings by varying the concentration of the drug-ethanol solution used. The release profiles of timolol from the gels are shown in FIG. 4 through FIG. 10 where the specific gel, its thicknesses, and the concentrations of drug-ethanol solution used to load the appliance are indicated in the Brief Description of the Figures

As can be clearly seen from FIG. 4 through FIG. 10, all gels release timolol over a long period of time. Also, where the quantity of timolol in the drug-ethanol solution was greater by an order of magnitude, the quantity released from the resulting gel at any given time was more than an order of magnitude greater. Furthermore, the total timolol release and the quantity released with time appear to be relatively independent of the gel thickness, particularly at early release times. The similar amounts released for all thicknesses at early release times is consistent with a releases rate that is controlled by diffusion when the diffusion boundary layer thickness is less than the thinnest of the gels under investigation. In most cases, for equivalent thicknesses, the greater the content of the macromer and the DMA in the formulation, the more timolol released over a given period of time. This indicates that the system can be tuned by the composition of the silicone hydrogels and/or by the concentration of the drug solutions used to load the drug. The initial burst is small in each system. Although small, burst release of the drug to the eye can be avoided by soaking the lens in a buffer solution for a sufficient time to extract sufficient amounts of the drug. In this manner any initial release is to a buffer solution rather than to the eye. One of ordinary skill in the art can appreciate that the compositions, loading conditions, and any extraction regiment can be modified to fit a desired release profile for the desired dosage form.

### Example 4

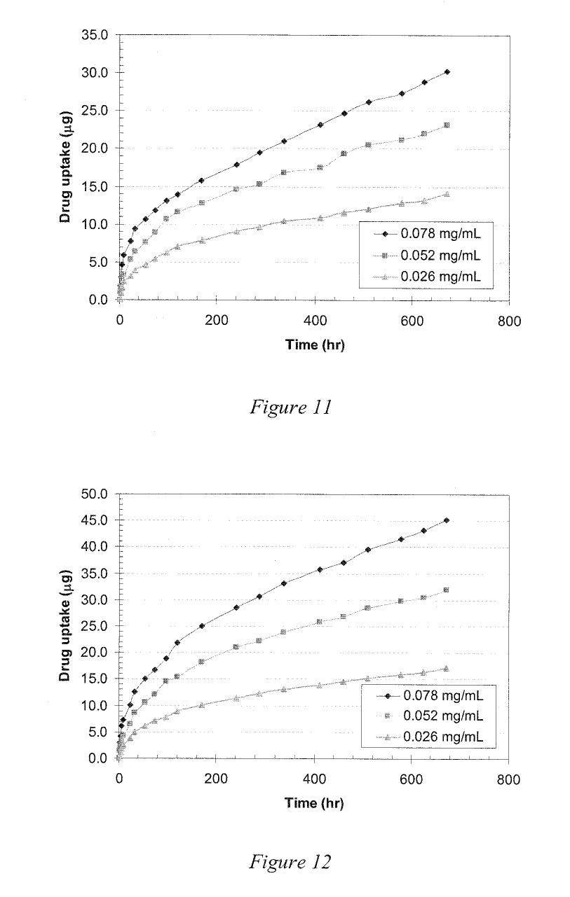

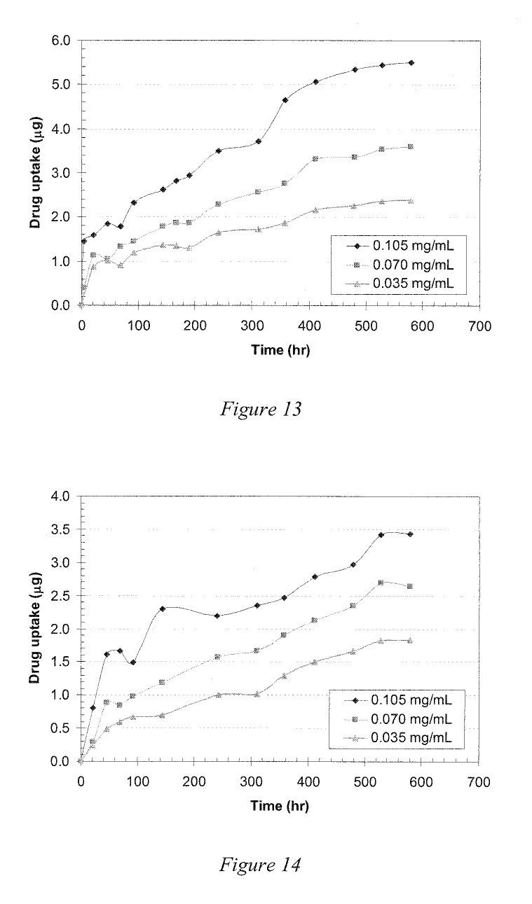

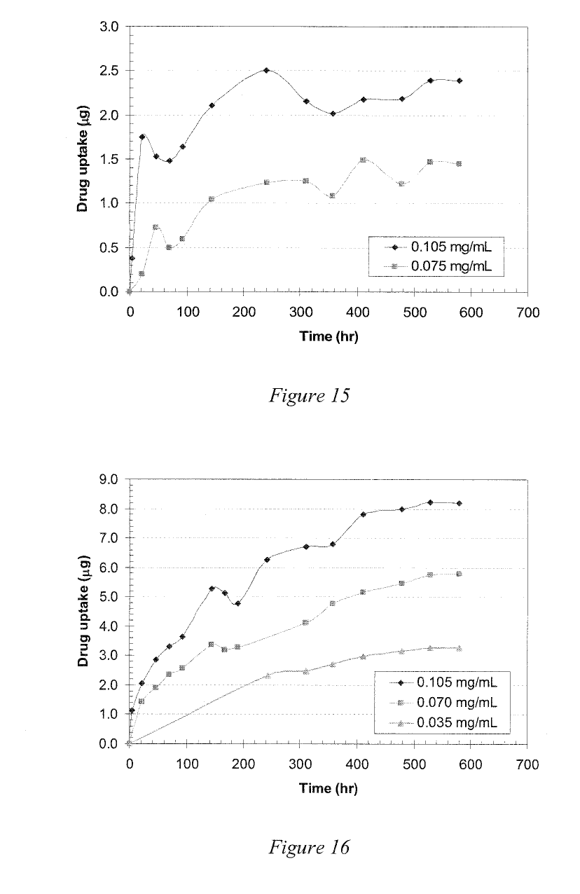

In contrast to the previous examples where drugs were loaded into the silicone hydrogel by soaking the gels in drug-ethanol solutions, studies were conducted in which the silicone hydrogels were soaked in aqueous drug solutions and the drug absorbed by the gel was determined over time by measuring the changes in the drug concentrations in the aqueous phase. These loading experiments were conducted for DX and timolol. The uptake profiles of DX and timolol from the aqueous solutions are shown in FIG. 11 through FIG. 16 where the gel type, its thicknesses, volume of aqueous drug solution and the concentrations of drug in the loading solutions are indicated in the Brief Description of the Figures.

### Example 5

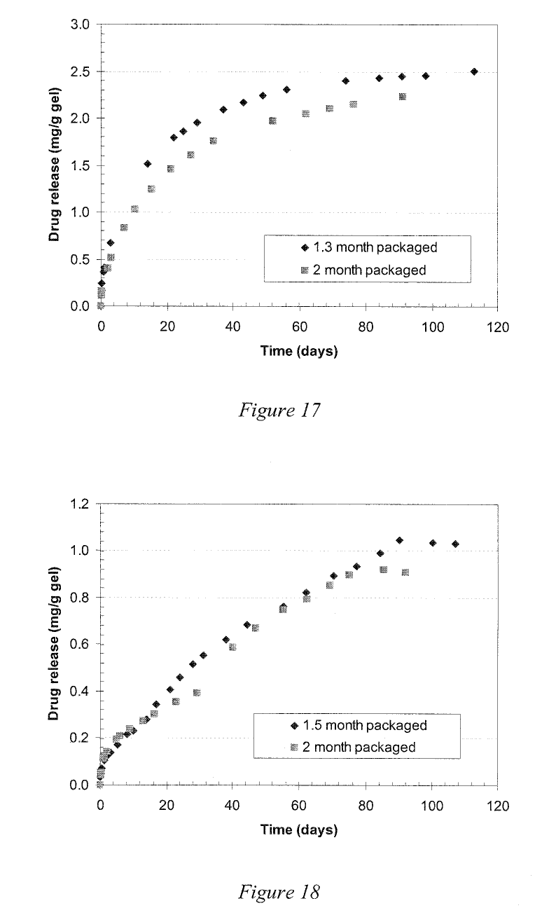

Commercial contact lenses are typically packaged in about 1-1.5 ml of packaging solution such as PBS. To estimate the effects of packaging on drug release behavior of silicone hydrogel appliances, packaging tests were conducted. A 0.1 mm thick silicone hydrogel was cut into circular pieces of 1.75 cm diameter and a drug was loaded by soaking the silicone hydrogel in a drug-ethanol solution. A 0.1 mm thick piece of GEL1 was loaded with a drug by soaking in drug-ethanol solution of 6.4 mg/ml for timolol and 4.8 mg/ml for DXA. The GEL1 pieces were then blotted with wipes to remove excess drug-ethanol solution from the pieces. The pieces were dried in air overnight before conducting the packaging tests. The silicone hydrogels were then stored in 1 ml of PBS (packaging) solution in a sealed vials for 1.3 or 2 months, and subsequently the appliance was placed in 2 ml of fresh PBS solution. The release of the drug over time is plotted in FIG. 17 and FIG. 18 for timolol and DXA, respectively. For timolol, about 364 μg and 404 μg of drug were lost to the packaging solution for the appliance stored for 1.3 months and 2 months, respectively. The residual drug released into the fresh PBS solution at an average rate of 0.09 mg/(g of gel·day) for 20 days, and then at a lower rate for another 2 months as shown in FIG. 17. DXA was released to a fresh PBS solution for an extended period of time, lasting longer than 90 days, at a rate of 0.3 μg/day, as shown in FIG. 18, after about 8 μg of drug was extracted in packaging solutions for both 1.5 months and 2 months of storage. Additional description of packaging studies as well as the preparation and properties of silicone hydrogel appliances for release of drugs according to the invention is found in Kim et al. Biomaterials, 2008, 29, 2259-69 and incorporated by reference herein.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1-35. (canceled)

36. A method of controlled release of bioactive agents from a silicone hydrogel appliance comprising:
providing a silicone hydrogel article;
absorbing at least one of said bioactive agents into said article to form said appliance; and
placing said appliance onto a tissue surface of a patient to be treated with said agent, wherein said agent partitions between said appliance and said tissue to release said agent from said appliance over a period of about 7 hours to about 6 weeks without a burst release of said agent.

37. The method of claim 36, wherein said release of said agent occurs over a period of about 7 hours to about 24 hours.

38. The method of claim 36, wherein said release of said agent occurs over a period of about 1 day to about 7 days.

39. The method of claim 36, wherein said release of said agent occurs over a period of about 1 week to about 6 weeks.

40. The method of claim 36, wherein said silicone hydrogel article comprises a copolymer of at least one siloxane containing monomer and at least one hydrophilic monomer.

41. The method of claim 40, wherein said siloxane containing monomers comprise a mixture of TRIS and an acrylate or methacrylate containing siloxane macromer.

42. The method of claim 40, wherein said hydrophilic monomers comprise a mixture of DMA and NVP.

43. The method of claim 40, wherein said hydrophilic monomers comprise about 10 to about 50 weight percent of said copolymer.

44. The method of claim 40, wherein at least one of said monomers provides functionality for specific interaction with one or more bioactive agents.

45. The method of claim 44, wherein said functionality is an ionic functionality, a metal complexing functionality, a multiple hydrogen bonding functionality, or a functionality that mimics a biological receptor for a bioactive agent.

46. The method of claim 36, wherein said step of absorbing comprises soaking said silicone hydrogel article in a solution of said agent for a sufficient period of time to achieve a desired level of said agent in said appliance.

47. The method of claim 46, wherein said solution comprises at least one solvent that swells said silicone hydrogel article.

48. The method of claim 47, wherein said solvent comprises a non-aqueous solvent.

49. The method of claim 48, wherein said non-aqueous solvent comprises ethanol.

50. The method of claim 46, where in the solution comprises an aqueous solution and wherein said period of time is about 1 to about 8 weeks.

51. The method of claim 46, wherein said agent comprises a drug.

52. The method of claim 51, wherein said drug is a non-ionic drug.

53. The method of claim 52, wherein said drug is selected from the group consisting of dexamethasone (DX), dexamethasone acetate (DXA) and timolol.

54. The method of claim 46, wherein said step of providing comprises:
providing at least one siloxane containing monomer, at least one hydrophilic monomer, and at least one polymerization initiator in a mold; and
activating said initiator wherein said article forms in the shape of said mold wherein said step of absorbing occurs prior to said step of activating said initiator.

55. The method of claim 46, wherein said article comprises a silicon hydrogel contact lens and wherein said tissue surface is an eye surface.

56. The method of claim 46, wherein said release from said appliance occurs over a period of about 7 hours to 6 weeks.

57. The method of claim 56, wherein said release from said appliance occurs over a period of 7 to 24 hours.

58. The method of claim 56, wherein said release from said appliance occurs over a period of 1 to 7 days.

59. The method of claim 56, wherein said release from said appliance occurs over a period of 1 to 6 weeks.

60. A sustained-release dosage form for administration of a bioactive agent to a tissue surface comprising a silicone hydrogel article containing said bioactive agent in a therapeutically effective amount with release to the tissue surface occurring over a period of about 7 hours to about 6 weeks.

61. The dosage form of claim 60, wherein said silicone hydrogel article comprises 20 to 80 weight percent silicone.

62. The dosage form of claim 60, wherein said silicone hydrogel article comprises a contact lens for ocular administration.

63. The dosage form of claim 60, wherein said bioactive agent comprises a drug.

64. The dosage form of claim 63, wherein said drug comprises a non-ionic drug.

65. The dosage form of claim 64, wherein said non-ionic drug is selected from the group consisting of dexamethasone (DX), dexamethasone acetate (DXA) and timolol.

66. The dosage form of claim 60, wherein said dosage form further comprises an aqueous solution wherein said article is contained in said solution prior to administration.

67. The dosage form of claim 66, wherein said solution contains a buffer.

68. The dosage form of claim 66, wherein said solution contains said bioactive agent.

69. The dosage form of claim 66, wherein said dosage form is provided with a printed date for earliest administration.
